(19) United States
(12) Patent Application Publication
(10) Pub. No.: US 2011/0251547 A1
(43) Pub. Date: Oct. 13, 2011

(54) TRANSFECTION WITH MAGNETIC NANOPARTICLES AND ULTRASOUND

(75) Inventors: James Xing, Edmonton (CA); Wiebing Lu, Edmonton (CA)

(73) Assignee: INTELLIGENTNANO INC., Edmonton, AB (CA)

(21) Appl. No.: 13/127,259

(22) PCT Filed: Nov. 9, 2009
(86) PCT No.: PCT/CA09/01629

§ 371 Date: May 3, 2011

Related U.S. Application Data
(60) Provisional application No. 61/112,451, filed on Nov. 7, 2008.

(51) Int. Cl. A61M 37/00; C12N 13/00

(52) U.S. Cl. 604/22; 435/173.4

(57) ABSTRACT

The invention includes a magnetic nanoparticle molecular delivery vehicle to be used for transfection and delivery of therapeutic molecules across cell membranes and to specific sites in the body, using magnetic forces and ultrasound.

### FIELD OF THE INVENTION

This invention relates to a nanostructured molecular delivery vehicle for delivering molecules to a selected site, and a method for transporting the molecular delivery vehicle across a biological membrane by applying a magnetic force and ultrasound.

### BACKGROUND OF THE INVENTION

Transfection is the introduction of foreign genetic material into eukaryotic cells using a vector as a means of transfer. The term transfection is most often used in reference to mammalian cells, while the term transformation is preferred to describe DNA transfer in bacteria and non-animal eukaryotic cells such as fungi, algae and plants.

Existing methods of transfection must overcome problems with the permeability of the cell membrane and the solubility of the transfected particle.

Drug delivery often involves transportation of the drug across cell membranes. The most basic method in vivo method is to introduce the drug into the blood stream by oral or intravenous methods and then hope it is absorbed by the correct cells. This non-discriminatory technique requires relatively large doses of the drug and simply does not work for some molecules such as DNA, which is used in gene therapy.

Existing methods to transfect material into a cell can be grouped into two categories: viral and non-viral. The utilization of viruses to transfect material into a cell has been shown to be extremely efficient; however, the possibility of a immune response to viruses and the insertion of mutagens into the body have proven to be serious concerns, especially in clinical trials. Non-viral drug delivery methods include naked DNA injection and electroporation. Unfortunately, naked plasmid DNA injection has shown to have a relatively low efficiency of gene delivery, while following electroporation tissue damage caused by the electric pulses has been observed.

Microinjection is a mechanical technique that utilizes a precision tool to place the molecule directly into the cell. This works excellently for DNA, however it is impractical in many situations as it can only be applied to one cell at a time.

A gene gun is a mechanical device that fires a particle bonded to the bio-molecule into the cell. These particles are relatively large and often damage cells. They also require large doses to be effective.

Electroporation is a physical method, which creates pores in the cell membrane by applying an electric shock to the cell. These pores allow the increased diffusion of materials into the cell. This increased permeability allows for easier transfection.

Sonoporation is similar to electroporation except it uses ultrasound to stimulate the cell membrane. The ultrasound also creates turbulence in the fluid surrounding the cell, which increases the rate of diffusion across the membrane.

Calcium phosphate transfection is a chemical method, which is very cheap. It uses calcium phosphate bonded to DNA. This molecule in some cases is able to transfect cells; however, this method is often ineffective and limited.

Viral delivery is a very effective method because viruses naturally are a carrier of genetic information and are very adept at entering cells. This makes them an obvious choice to help deliver DNA molecules into cells. However, the use of viral vectors is sometimes undesirable because of their immunogenicity and their potential mutagenicity. Furthermore, viral delivery is non-specific and can trigger side effects in the host.

Yet another method uses magnetic force and a molecular delivery vehicle to cross the cell membrane. A version of this method is described in United States Patent Application 2007/0231908 A1, and requires that the molecular delivery vehicle be oriented before it penetrates the biological membrane.

For most of the above methods, the effectiveness is extremely variable depending on the cell type being transfected. Some cells are known to be harder to transfect then others and these are usually the cells that hold the greatest reward.

Therefore, there is a need in the art for methods of transporting biomolecules and other molecules of interest into cells which mitigate the difficulties of the prior art.

### SUMMARY OF THE INVENTION

The present invention provides for transfection of cells using nanoparticles and magnetic forces to direct the nanoparticles through a cell wall or membrane. In one embodiment, the nanoparticle is directed through a cell membrane, a nuclear membrane, or a cell membrane in vivo such as the blood-brain barrier. In one embodiment, the invention further comprises the use of ultrasound to increase the permeability of the biological membranes. This results in greater efficiency or molecular delivery or transfection.

This invention comprises the following aspects (a) a method of creating nanoparticles, which are nontoxic, magnetic, and bondable to biological molecules or other molecules of interest; (b) a method of bonding such molecules to this nanoparticle; and (c) a system to force these nanoparticles through a membrane using a magnetic field. In one embodiment, ultrasound in the form of low-intensity pulsed ultrasound (LIPUS) is used increase the permeability of the membrane.

In one aspect, the invention comprises a method of delivering a molecule across a cell membrane using a delivery vehicle comprising a magnetic nanoparticle, the method comprising the steps of:
(a) fixing the molecule to the nanoparticle;
(b) positioning the nanoparticle in the immediate vicinity of the cell membrane;
(c) subjecting the nanoparticle and cell membrane magnetic field; and
(d) simultaneously subjecting the nanoparticle and cell membrane to ultrasound.

The nanoparticle comprises bonding sites so that the molecule can be attached to this nanoparticle. The number of bonding sites is variable as is the spacing between bonding sites. In addition, the type of bond may be covalent, ionic or another bond which is capable of fixing the molecule to the nanoparticle. In one embodiment, the molecule may comprise a genetic material such as DNA or RNA, proteins, or any other biological molecule.

The nanoparticle may comprise nanotubes, such as carbon nanotubes, or single-walled carbon nanotubes. In one embodiment, the nanoparticles may be biodegradable or biocompatible, and may comprise silica. The nanoparticles may display low or no toxicity to cells in vivo or in vitro.

On a macroscopic scale, this magnetic force can be used to control the molecular delivery vehicles to move to certain parts of a body. On a microscopic to nanoscale level, this force can be used to force the molecular delivery vehicles through a biological membrane. If necessary or desired, the molecular delivery vehicle can be further transported into the nucleus of the cell by moving it with a magnetic force.

This membrane may be the cell wall or the wall of the nucleus inside the cell, or another biological membrane such as the mitochondrion's double membrane. This membrane could also be the blood-brain barrier. Thus, this invention may allow for the transportation of molecules into the central nervous system.

Thus using this method, a bio-molecule can be delivered to a specific target.

In one embodiment, the invention comprises a molecular delivery vehicle which comprises a nanostructure which is magnetic and has bonding sites so that a bio-molecule can be attached to this molecular delivery vehicle. The number of bonding sites is variable as is the spacing between bonding sites. In addition, the type of bond may be covalent, ionic or another bond which is capable of holding the biomolecule.

Using this magnetic force the magnetic nanoparticle can be controlled in numerous ways. In one embodiment, the delivery vehicles can be collected in one location using a magnetic force that attracts to that location, such as an organ or specific tissue in vivo.

In one aspect, the invention comprises a method for using the molecular delivery system to deliver molecules into cells or transfect such cells in vitro or in vivo. In vitro cells may be supported on solid or liquid media.

### BRIEF DESCRIPTIONS OF THE DRAWINGS

In order that the above-recited and other features and advantages of the present invention will be readily understood, a more particular description of the invention is given. Specific examples thereof are detailed, the result of which are illustrated in the appended figures. Any example is only a single embodiment of the invention, and is not to be considered in any way the limit of its scope. In the accompanying figures:

FIG. 1A is a sketch of a magnetic single walled nanotube and FIG. 1B is a sketch of a spherical magnetic nanoparticle after it has been functionalized.

FIG. 2 shows the delivery vehicle being forced though the cell membrane. The arrows depict the magnetic field. In this depiction the carbon nanotube is being used for the delivery.

FIG. 3 depicts the use of a magnet to collect the nanoparticles at a certain location in the body. In this case the particles are being collected at the top of the patients left arm.

FIGS. 4A, 4B, 4C, and 4D show schematic processes for functionalizing a single-walled nanotube.

FIG. 5A and 5B show XPS and IR spectra for carboxylated SWNTs.

FIGS. 6A and 6B show IR and UV-vis spectra for FITC labelled SWNT. The vertical axis A shows absorption.

FIG. 7A shows a confocal microscopy image showing control cells. FIG. 7B shows cells a confocal microscopy image showing cells with FITC labelled nanoparticles in the cytoplasm. FIGS. 7C and 7D show confocal microscopy of MCF-7 control cells and cells transfected with nanoparticles bound with GFP plasmid.

FIG. 8A show distribution of FITC labelled nanoparticles in control MCF-7 cells and FIG. 8B shows distribution in MCF-7 cells exposed FITC labelled magnetic nanoparticles and a magnetic field.

FIG. 9 shows a graph of percentage uptake by MCF-7 cells.

FIGS. 10A, 10B, and 10C show FITC labelled nanoparticles delivered into hematopoietic stem cells in a control, after 3 hours and after 6 hours, respectively.

FIG. 11 shows a graph demonstrating viability of MCF-7 cells after FITC labelled nanoparticle uptake compared to control cells.

FIG. 12A shows FACS results for Negative control sample contained no GFP plasmid, no Definity, and was not sonicated. FACs results: Marker: M1, % Gated: 0.16. Extremely high cell viability is observed. FIG. 12B shows FACS results for Positive control sample contained 2 μg of GFP plasmid, no Definity, the lipofection agent PEI, and was not sonicated. FACs results: Marker: M1, % Gated: 33.12%. Very low cell viability is observed.

FIG. 13 shows FACS results FACs results for sample sonicated at 0.5 W/cm2, with a 20% duty cycle for 60 seconds. DNA plasmid concentration was varied. FIG. 13A—DNA plasmid concentration: 2 μg/mL, marker: M1, % Gated: 16.20. FIG. 13B—DNA plasmid concentration: 15 μg/mL, marker: M1, % Gated: 26.93. FIG. 13C—DNA plasmid concentration: 30 μg/mL, marker: M1, % Gated: 32.51. A high amount of cell viability is seen in all cases.

FIG. 14 shows FACs result for sample sonicated at 0.3 W/cm2, with a 100% duty cycle for 60 seconds. DNA plasmid concentration was 30 μg/mL. FACs results: marker: M1, % Gated: 14.67. Cell viability is observed to have decreased.

FIG. 15 FACs result for sample sonicated at 0.5 W/cm2, with a 100% duty cycle for 60 seconds. DNA plasmid concentration was 30 μg/mL. FACs results: marker: M1, % Gated: 32.12. Cell viability is observed to be low.

FIG. 16 shows a picture of a biocompatible silica nanotube.

FIG. 17 shows a graph of IR spectra of Si-NT which has been carboxylated.

FIG. 18 shows HeLa cells which have been transfected with Si-NT-GFP plasmid, compared with a control.

FIG. 19 shows a graph demonstrating low toxicity of the Si-NT after 48 and 72 hours of incubation.

### DETAILED DESCRIPTION OF PREFERRED EMBODIMENTS

This invention comprises a method to deliver biomolecules or other molecules of interest into cells using a a molecular delivery vehicle, which is magnetically drivable and capable of bonding to at least one bio-molecule. This molecular delivery vehicle can pass through the cell wall with the aid of an external magnetic force.

“Biomolecule”—a biological molecule that performs some function which influences the behavior or nature of a biological system.

“Magnetic nanoparticle”—any particle on the nanoscale (having one dimension less than about 100 nm) the motion of which is influenced by a magnetic field.

“Nanoscale”—the range of lengths used to measure objects from 0.1 nm up to 1000 nm where 1 nm is 10⁻⁹ meters.

“Transfect”—a process to introduce foreign genetic material into a cell.

The present invention relates to the use of magnetic nanoparticles to transport biomolecules and other molecules of interest across a cell membrane.

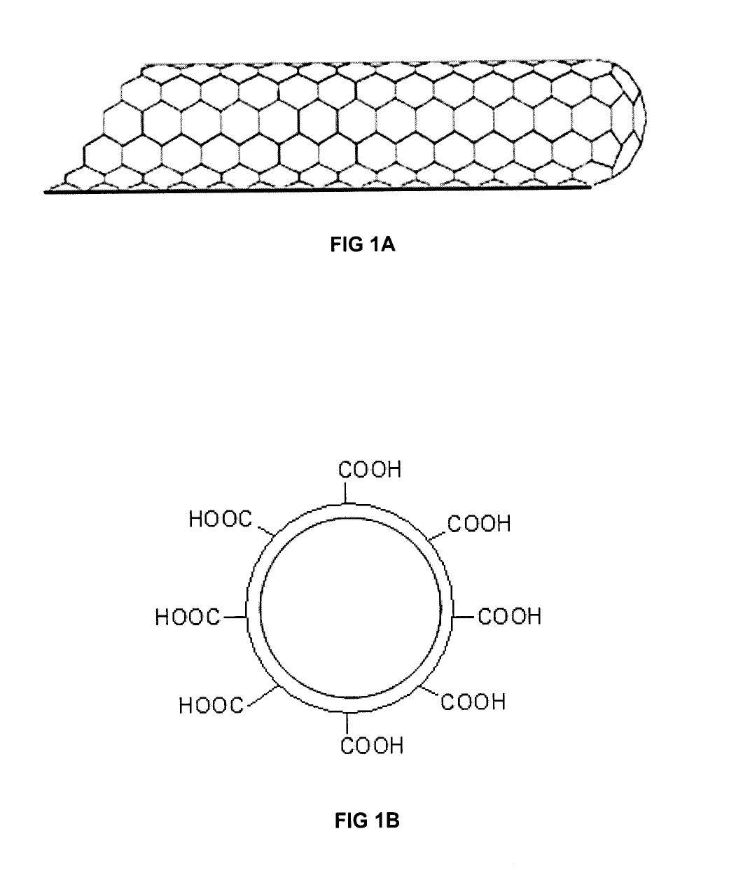

In one embodiment of the present invention, the magnetic nanoparticles take the form of a metal core coated in a material such as carbon as shown in FIG. 1B. These nanoparticles are then functionalized so that a bio-molecule can be bonded to them.

In one embodiment of the present invention, the magnetic nanoparticles are carbon nanotubes, such as single-walled carbon nanotubes (SWNT) embedded with magnetic metal atoms (FIG. 1A). In one embodiment, the magnetic metal atoms comprise nickel, iron or cobalt.

Single-walled carbon nanotubes are well known in the art and may be synthesized using any suitable technique, such as chemical vapor deposition technique (CVD). These carbon nanotubes are grown from a surface using nickel or yttrium, or both nickel and yttrium, as seed. In one embodiment, the nickel and/or yttrium is thus incorporated at least into the tip of the carbon nanotube. In one embodiment, suitable SWNTs have a diameter between about 1.2 to about 1.5 nm, and a length of about 2 to about 5 μm. The SWNTs may be either armchair or chiral nanotubes. In one embodiment, the SWNTs used are armchair (5,5) nanotubes.

The magnetic nanoparticles or carbon nanotubes are prepared for bonding to a bio-molecule by adding functional groups to them. These functional groups act as the bonding site, which will hold the bio-molecule to the nanoparticles or the carbon nanotubes. In addition, functionalization is important as many nanoparticles or carbon nanotubes, particularly SWNTs, are insoluble in water. Functionalization increases their water solubility.

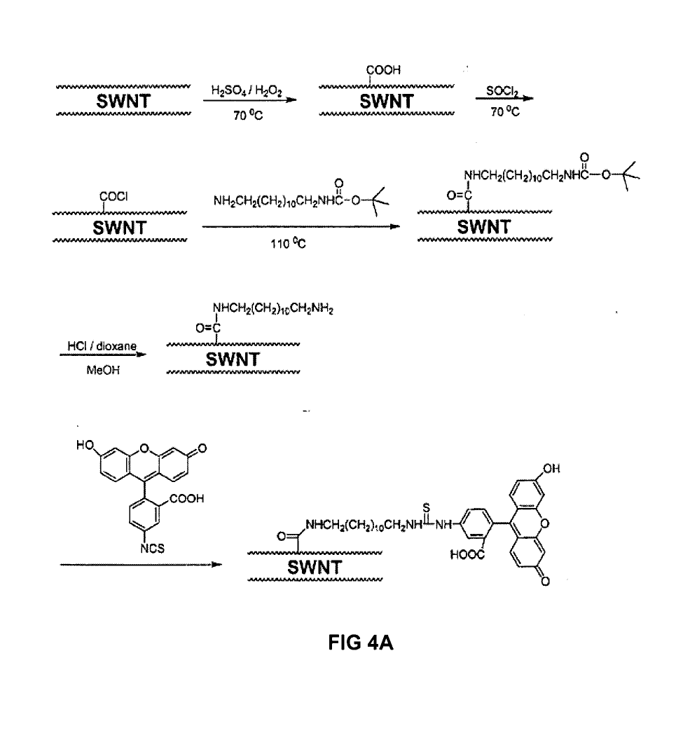

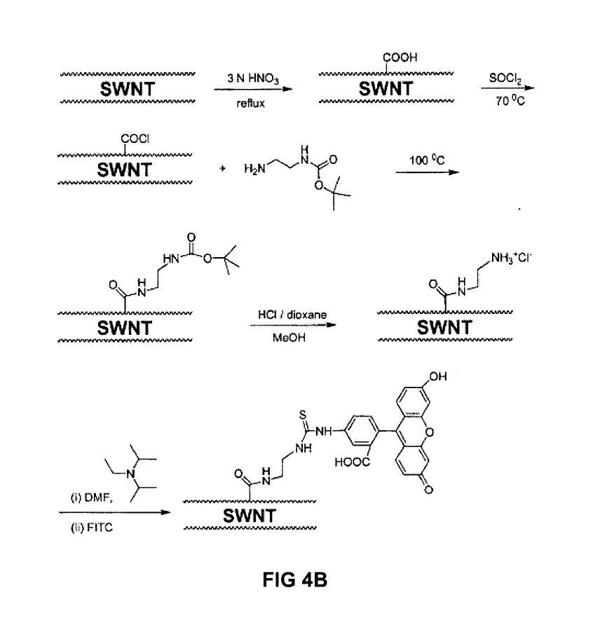

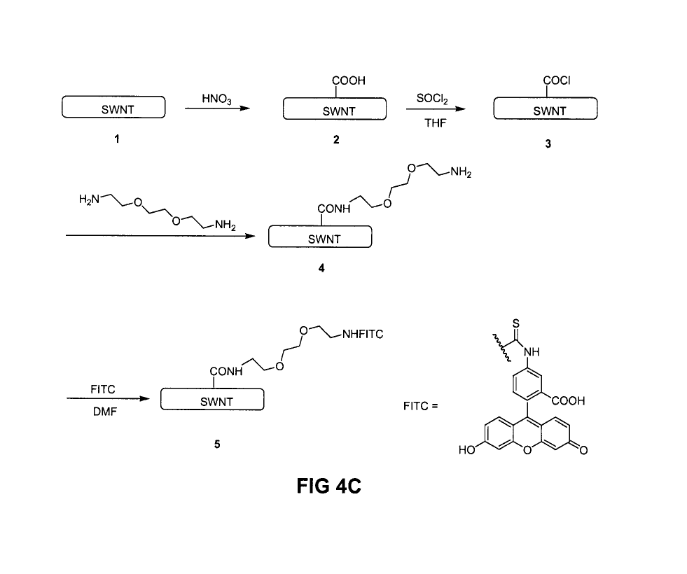

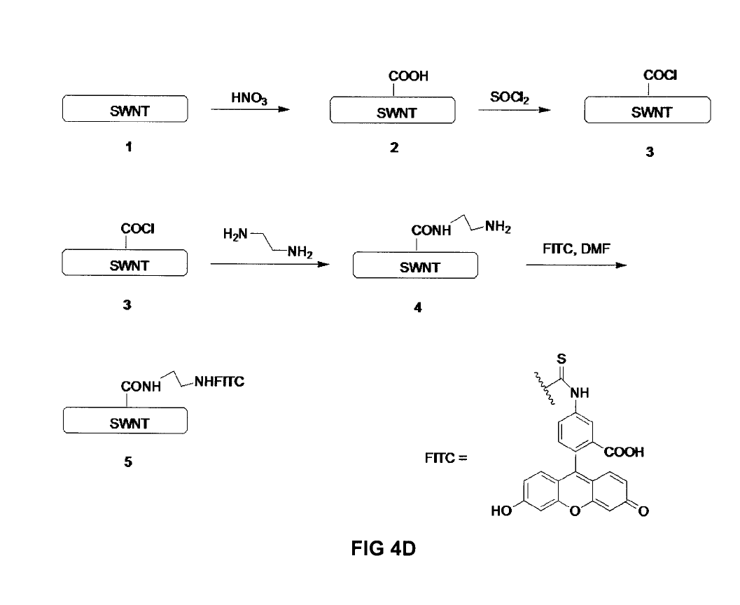

In one embodiment, shown schematically in FIGS. 4A and 4B, functionalization is achieved by chemically altering the surface of the carbon nanotube. In one example, the surface of the magnetic carbon nanotube is carboxylated and the carboxylic acid is reacted with thionyl chloride to provide an acid chloride. The acid chloride may then be coupled with tert-butyl-12-aminododecylcarbamate, or tert-butyl (2-aminoethyl) carbamate, followed by deprotection of the Boc group to provide the amine derivative.

In an alternative embodiment, amine derivative nanotubes can be produced by reacting the acid chloride nanotube with then 2′-(ethylenedioxy)bis(ethylamine) to produce the amine derivative, as shown in FIG. 4C. In a further alternative, the amine derivative may be formed using ethane—1,2 diamine, as shown in FIG. 4D.

In one example, the amine derivative is then reacted with fluoroscein isothiocynanate (FITC) giving rise to the FITC derivatized magnetic carbon nanotube.

These magnetic carbon nanotube bonded molecules may then be subjected to a magnetic field and a cell culture, as described herein.

Biomolecules such as DNA or RNA can be attached to carboxyl functional groups on the surface of the nanoparticle or carbon nanotube. In one example, plasmid vectors may be combined with carboxylated SWNTs and 1-ethyl-3-(3-dimethylaminopropyl) carbodiimide (EDC) in 2-[N-morpholino]ethane sulfonic acid (MES) or a phosphate buffer (pH 4.5) for the aminization between the primary amine groups in the DNA molecules and carboxylic groups on the nanotubes. Alternatively, DNA or RNA can be bound by electrostatic interaction with amine functional groups on the surface of the nanoparticle.

The nanoparticles may comprise silica or other materials which may be biodegradable or biocompatible within a cell, such as, without limitation, nanocellulose, or nanocrystalline cellulose. The term “biodegradable” as used herein means that the substance may be broken down into innocuous products by the action of living things. The term “biocompatible” means that the substance does not have toxic or injurious effects on biological function of cells either in vitro or in vivo. In one embodiment, a carbon nanotube may be coated with silica and the carbon then removed or burnt out, leaving a silica nanotube based on the carbon template. The silica nanotube may then functionalized using methods similar to those described herein for carbon nanotube, and as are known to those skilled in the art.

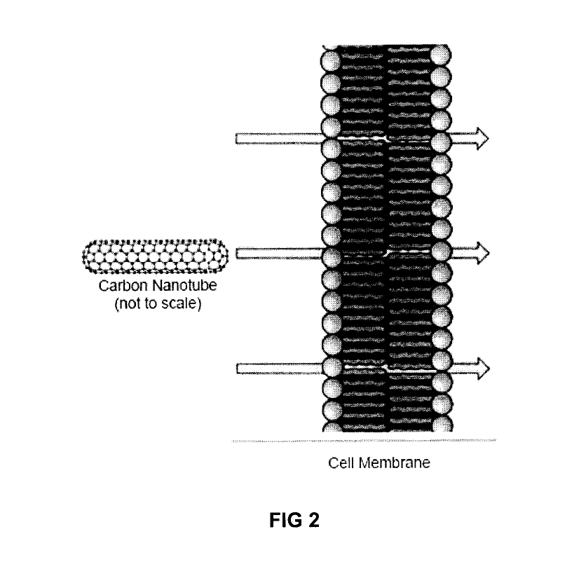

Once the biomolecule or other molecule of interest is bonded to the magnetic nanoparticle, the nanoparticle is placed in a solution along with the cells that are to be transfected and a magnetic force is applied so that the nanoparticles are accelerated through the solution. Inevitably, these will collide with a cell and there will be a probability that the particle will pass through the membrane into the cell, as shown schematically in FIG. 2. If the particle does not enter the cell, it will be free to accelerate again to attempt to transfect another cell. A substantial majority of the cells will be transfected after a relatively short period.

The magnetic field that is used to drive the molecular delivery vehicles is configured so that it provides a magnetic force which can be static or variable in direction and magnitude. In one embodiment, the magnetic field is configured so that the magnetic force can change between being variable and static at different stages of delivery. In one embodiment, the magnetic nanoparticles can be caused to move in complex paths by constantly varying magnetic force, which is changing its magnitude and direction.

In another embodiment, the delivery vehicles can be moved in complex paths and at variable velocities and accelerations.

In one embodiment, the membrane that must be transfected can be made more permeable by applying ultrasound energy to the cell culture, such as low-intensity pulsed ultrasound. The ultrasound may be applied at higher frequencies than is known to enhance cell growth. Typically LIPUS has been used at frequencies less than about 1 MHz, however, in embodiments of the present invention, any frequency between 1 MHz to 2 MHz may be used, such as 1.5 MHz.

Ultrasound can be applied using conventional or slightly modified therapeutic ultrasound transducers. The intensity of the ultrasound energy may vary from 0.1 W/cm² to about 1.0 W/cm². In one embodiment, the intensity is between about 0.3 W/cm² to about 0.5 W/cm². Varying duty cycles and pulse repetitions may be used, such as a duty cycle between about 20% and 100% and a repetition frequency of 100 Hz. In general, higher intensities and longer duty cycles will increase movement across cell membranes, at the expense of cell viability. Total ultrasound energy, calculated as follows, should preferably not exceed a level where cell viability is substantially impaired.

Energy(J)=Intensity*Duty Cycle*Time

In one embodiment, total energy may optimally be 18000 mJ.

Suitable ultrasound contrast agents, such as Definity™ (Bristol-Myers Squibb) may be used to promote microcavitation in the vicinity of the cells.

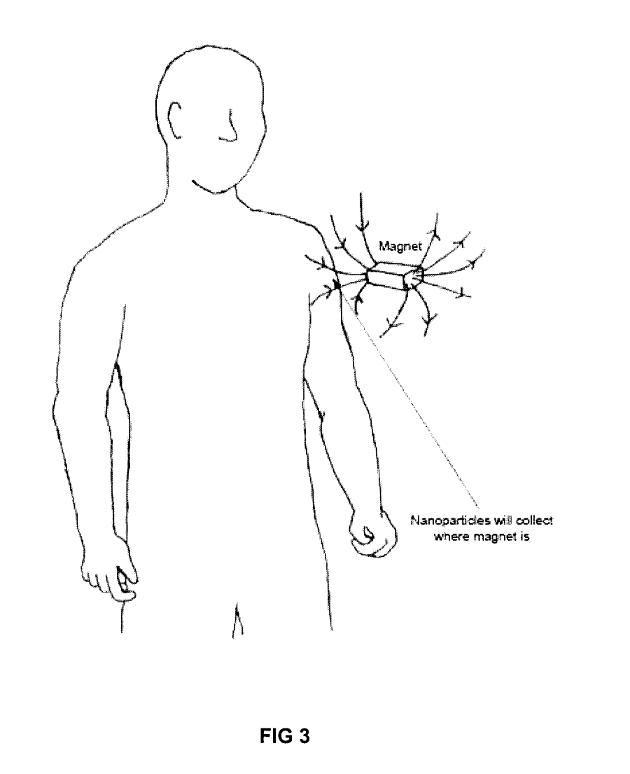

In one embodiment, the magnetic nanoparticles may be used in vivo to deliver therapeutic agents such as drugs or biomolecules to a specific target. A magnet may be placed at the site where the magnetic nanoparticles are to be focused, as shown in FIG. 3. As the magnetic nanoparticles circulate through the body, they will accumulate at the site where the magnet is located. Thus, the nanoparticles deliver the biomolecules to a specific target region.

In one embodiment, this targeted delivery mechanism may be used to deliver molecules into difficult to access areas, such as across the blood-brain barrier into the central nervous system. The magnetic nanoparticles can be collected at a specific site of the blood brain barrier using a magnetic field. Then, using a magnetic force these nanoparticles can be forced across the barrier.

Once the nanoparticles have been concentrated at a specific point or region, the nanoparticles can be forced into cells at that site by using a magnetic force with rapidly alternating direction. This will excite the particles to move back and forth quickly. As they move around they will collide with the cell membrane and at least a portion of the particles will pass through the membrane into the cell. In one embodiment, the use of ultrasound and magnetic forces may be used to enhance such movement in vivo. Ultrasound transducers which apply ultrasound energy to the human body are well known for imaging purposes, and may be used for the molecular delivery systems described herein with little or no modification.

The present invention may be embodied in other specific forms without departing from its structures, methods, or other essential characteristics as broadly described herein and claimed hereafter. The described embodiments are to be considered in all respects only as is, therefore, indicated by the appended claims, rather than by the foregoing description. All changes that come within the meaning and equivalence of the claims are to be embraced within their scope.

### EXAMPLES

The following examples are intended to be illustrative of the described invention, and not be limiting of the invention claimed herein, except where specifically recited.

### Example 1

### Synthesis of FITC-Labelled Carbon Single-Walled Nanotubes (SWNT) (Scheme Shown in FIG. 4B)

Nickel containing carbon nanotubes were refluxed with 3N HNO₃ for 45 h to introduce carboxylic acid groups. After refluxing, the solution was diluted with deionized water, filtrated and washed several times with deionized water. The acid treated SWNTs were collected and dried under vacuum.

100 mg of SWNTs were stirred in 20 mL of SOCl₂ (containing 1 mL of dimethylformamide) at 70° C. for 24 h. After centrifugation, the brown-colored supernatant was decanted and the remaining solid was washed with anhydrous tetrahydrofuran. After centrifugation, the pale-colored supernatant was decanted. The remaining solid was dried under vacuum.

A mixture of the resulting SWNTs and 1 g of tert-butyl-2-aminoethylcarbamate was heated at 100° C. under an argon atmosphere for 100 h. After cooling to room temperature, the excess tert-butyl-2-aminoethylcarbamate was removed by washing with methanol. The resulting black solid was dried under vacuum.

The coupling product of SWNTs with tert-butyl-2-aminoethylcarbamate was suspended in MeOH and a solution of HCl in dioxane (4 N) added, the resulting mixture was stirred at room temperature for 4 h. Then anhydrous ethyl ether was added, the resulting precipitate was collected and dried under vacuum.

The amine groups-containing SWNTs were suspended in a mixture of DMF and diisopropylethylamine and a solution of fluoroisothiocyanate (FITC) in DMF was added. The resulting mixture was stirred for 4 h at room temperature in darkness. Then anhydrous ethyl ether was added, the resulted precipitate was collected by centrifugation and washed thoroughly with ethyl ether and methanol, dried under vacuum to give FITC-labeled SWNTs.

In an alternative method, shown schematically in FIG. 4C, SWNTs from Aldrich were oxidized to form carboxylic acid groups on the surface. These nanotubes were reacted with thionyl chloride and then 2′-(ethylenedioxy)bis(ethylamine) to produce amine-terminated nanotubes. The amine was then reacted with FITC to attach FITC to SWNTs.

### Example 2

### IR, XPS and UV-vis Characterization

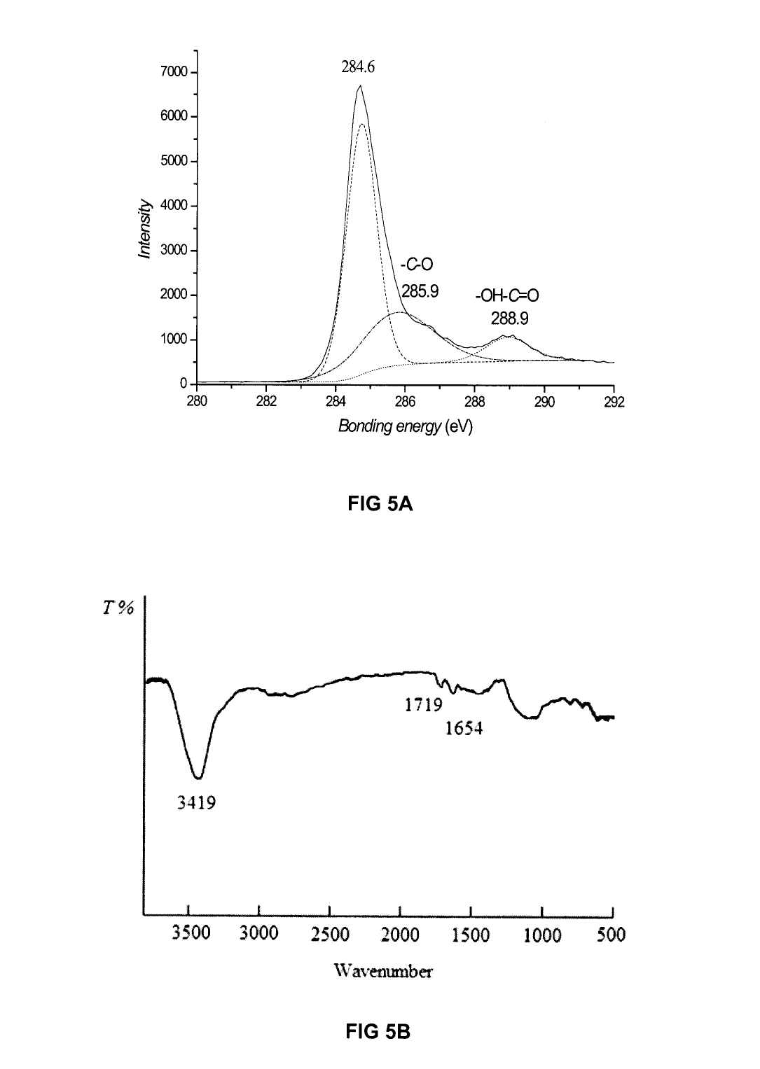

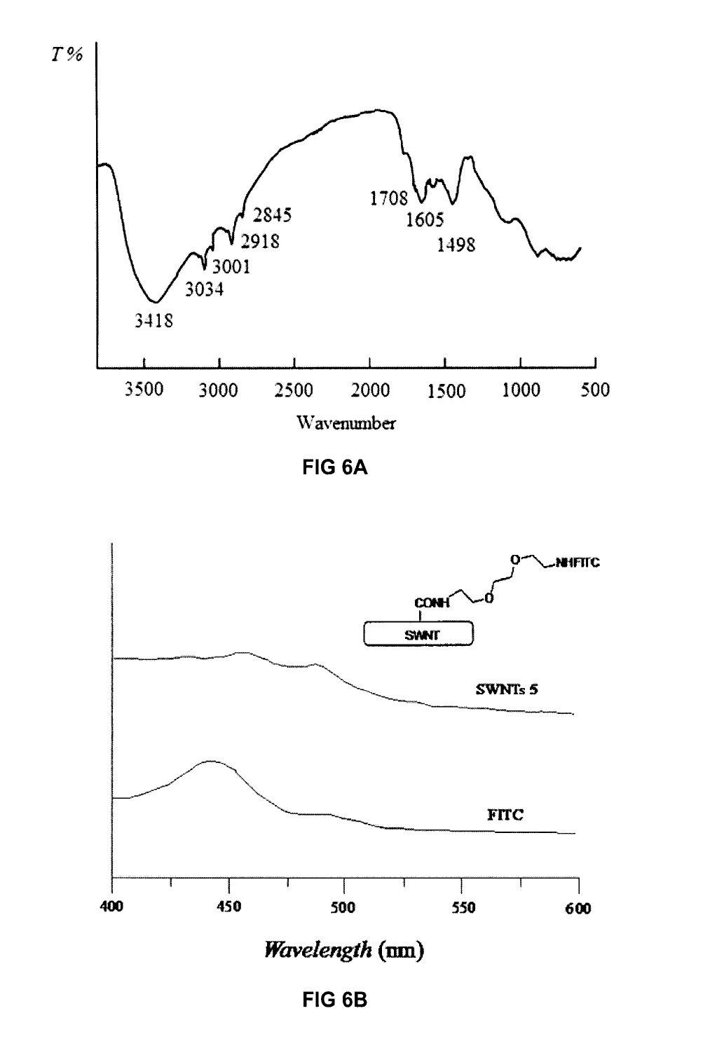

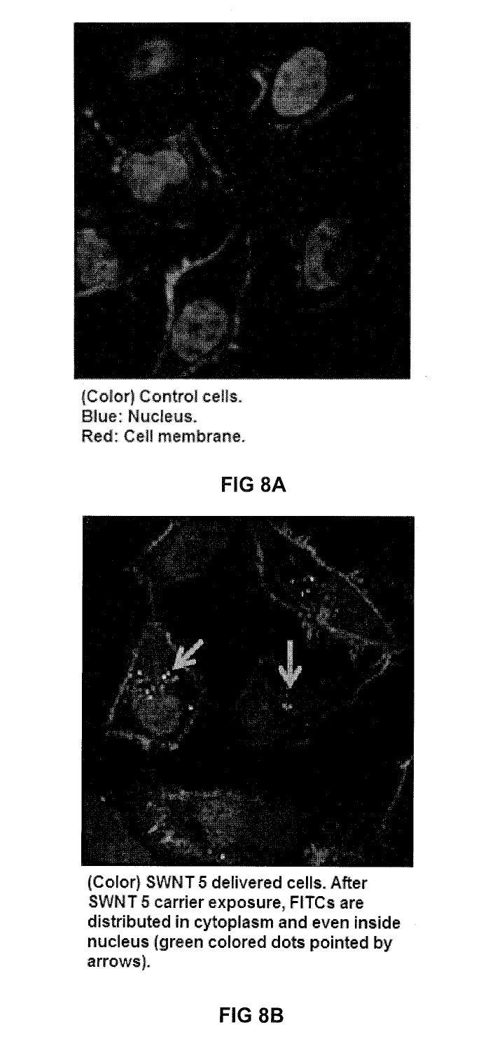

To validate the all synthesis take place, all of the intermediates shown in FIG. 4C and final product (SWNT-FITC) were characterized by Infrared (IR), X-ray photoelectron spectroscopy (XPS) and UV-vis and the results are shown in FIGS. 5 and 6. IR data clearly show that SWNTs were successfully functionalized to give carboxylic groups and XPS data show that about 6.1% of the carbon atoms are present as carboxyl groups. The UV-vis spectrum of the FITC-labeled SWNT in water is shown in FIG. 8, for comparison, the UV-vis spectrum of the FITC in water is shown in the same figure.

### Example 3

### Fluorescent Staining and Imaging

FITC-labeled SWNTs (CNT-FITC) as prepared using the method described in Example 1 (FIG. 4B) were used to stain and image a human breast adenocarcinoma cell.

Materials
Cell—MCF-7
Medium—GIBCO 11330, DMEM/F12 (1:1)
Formaldehyde Solution(w/v) 16%, Methanol-free, Pierce, Cat# 28906
Hoechst—Invitrogen Cat# 33342
Rhodamine Phalloidin—Invitrogen Cat# R-415
   (Rhodamine Phalloidin 300 U was dissolved in 1.5 ml Methanol to form concentration of 200 units/ml, distributed them into 10 ul each vial, store at −20° C.)
PBS buffer
Block buffer—PBS/0.5% BSA
Magnets—Applied Magnets Cat#ND075 (www magnet4less.com) 2×1 in thick disc, Grade N42, Rare earth Neodymium super strong magnet (Pull force: 176 lbs)

Round cover slips were placed into a 6-well or 24-well plate, one cover slip into one well and MCF-7 cells into each well, cell number: 1×10⁵/ml, and incubated at 37° C. over night. Add Hoechst into each well (1 ul Hoechst in 1 ml medium) and incubate at 37° C. for 1 h. 1 ml of CNT-FITC was added into each well of the plate (except the control) and incubate at 37° C. for 1 h. Each well was washed 3 times with PBS.

A cover slip picked out of one well with tweezers, and vertically inserted into a beaker containing 10 ml serum-free medium supplemented with CNT-FITC (10:1, medium: CNT-FITC) was placed on hotplate (magnetic stirrer) with the cells facing the incoming nanotubes for 3 min. The speed of the stirrer was set at 1,200 rpm. The cover slip was laid on one dish containing serum-free medium without CNT-FITC, and the dish was placed on a magnet for 7 min. The cover slip was then washed 3 times with PBS and placed in another 24 well plate, along with cover slips which were not placed on a magnet.

The cells were fixed with 4% Formaldehyde Solution for 10 min (or over night at 4° C.). The formaldehyde solution was removed and the cells washed 3 times with PBS. 250 ul of PBS/0.2 TX-100 was added onto the cover slips in the wells and place at room temperature for 10 min. Again the cells were washed 3 times with PBS, and blocked with 250 ul of PBS/0.5% BSA for 20 min. 2.5 ul Rhodamine Phalloidin was added to 50 ul block buffer and the mix pipetted on parafilm. The cover slip was overlaid onto the solution in place for 30 min

The cover slips were then placed back to the plate and washed 3 times with PBS. The coverslips were then mounted onto slides and send for the confocal microscopy. Samples were imaged with a laser scanning confocal microscopy 510 (Carl Zeiss) equipped with Axiovert 100M microscopy (Zeiss), a F-Fluar 40X-1.3 NA oil lens and 3 different lasers (Uv, Argon/2 and HeNe1).

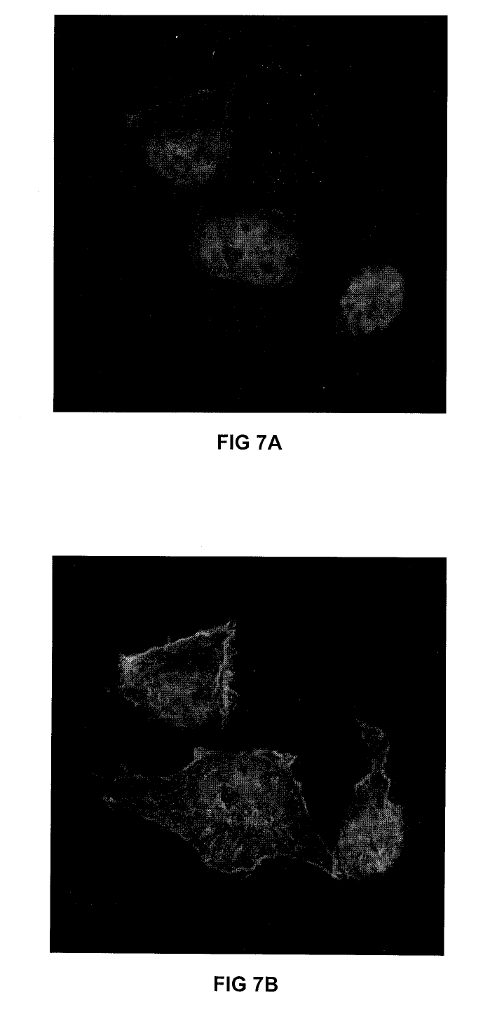

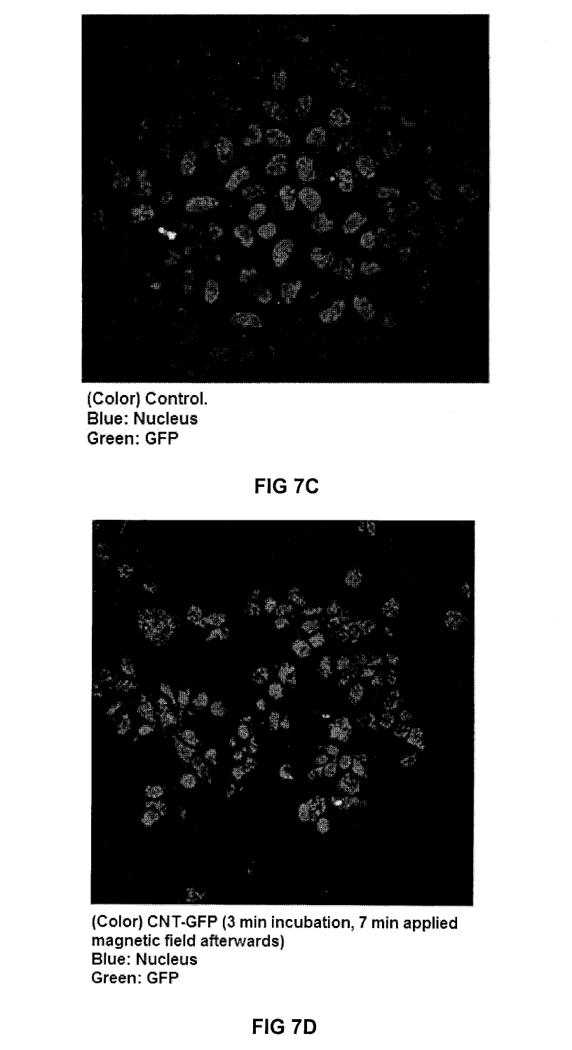

As shown in FIGS. 7A and 7B, the cell nuclei fluoresce strongly as a result of the Invitrogen stain which combines with double-stranded DNA. In FIG. 8B, fluorescence of the FITC moities may be plainly seen within the cells cytoplasm, indicating that the CNT-FITCs have passed through the cell membranes and into the cytoplasm.

In another example, SWNT were conjugated to GFP plasmid (pDRIVE5-GFP) by covalent bonding using EDC and a phosphate buffer. The SWNT-GFP plasmid was then incubated with MCF-7 cells for 3 min, followed by 7 minutes with a magnetic field supplied by a magnetic stirrer. The cells were then incubated for 24 hours and confocal microscopy was used to confirm GFP expression. FIG. 7D shows results of GFP fluoresence within the cells, as compared to the control cells in FIG. 7C.

### Example 4

### Cell Uptake Efficiency

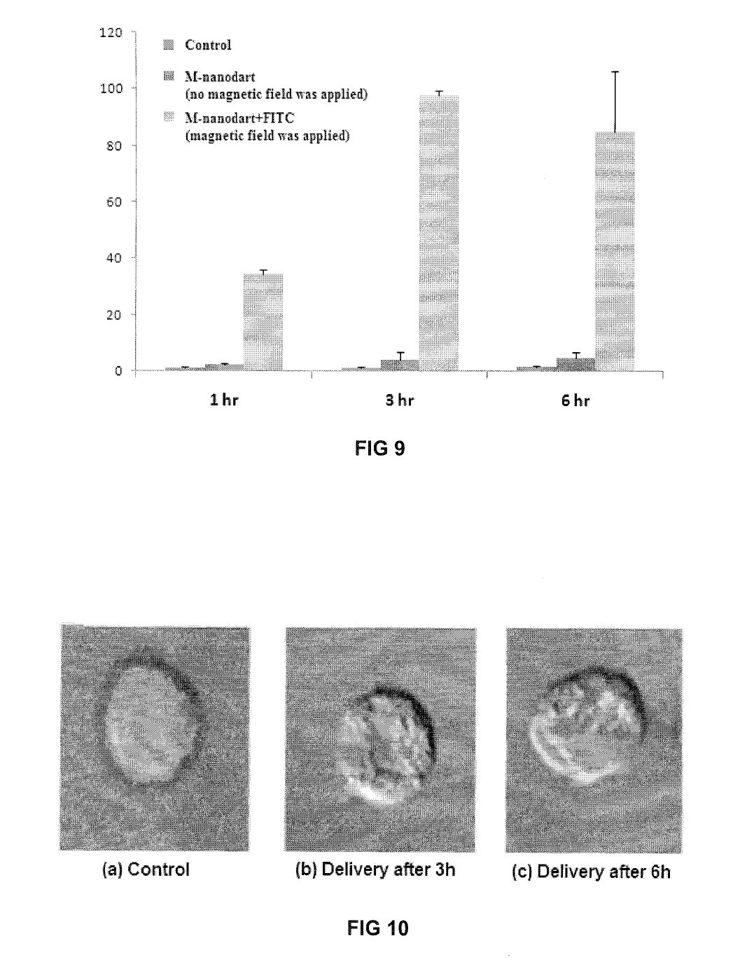

FITC-labeled SWNT was delivered into adherent MCF-7 breast cancer cells. Following the delivery and recovery phases, the fluorescently-labelled SWNT was detected by confocal microscopy. The results are presented in FIGS. 8A and 8B. The data clearly shows that the SWNT crossed the cell membrane and entered the cell cytoplasm and even into the nucleus (refer to the green dots in FIG. 8B; some of them are pointed by the arrows). The uptake rate is about 90% shown in FIG. 9.

In addition to delivery of FITC to adherent cells, like MCF-7 cells, we also successfully delivered FITC into difficult-to-transfect cells, or suspension cells, like hematopoietic stem cells (HSCs). FIG. 10 shows the delivery results. The results show that SWNT can deliver FITC into HSCs. As time increases to 3 and 6 hours, more FITC enters the cell (FITC shows as green fluorescence). The control sample showed no internal fluorescence.

### Example 5

### Cell Viability

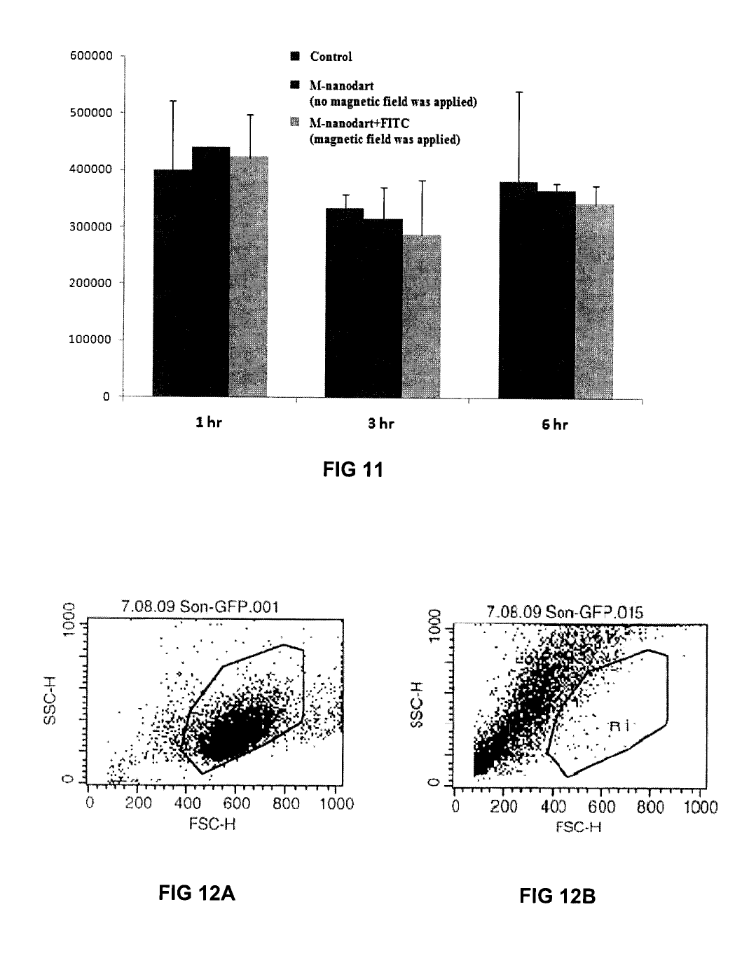

Furthermore, it is worth noting that cell viability was not compromised by SWNT uptake when compared with control, as shown in FIG. 11. Viability of MCF-7 cells after FITC-SWNT uptake with exposure to a magnetic field was compared to the control cells and cells exposed to SWNT alone with no magnetic field. Cells exposed to SWNT appear to substantially similar to control populations for viability after 6 hours.

### Example 6

### Ultrasound Delivery (USD)—Cell preparation and DNA

USD and transfection was assessed using human breast adenocarcinoma cells (MCF-7). Cells were maintained in the IMDM medium with 10% fetal bovine serum. Cells were harvested a day before the experiment by adding 0.25% Trypsin to the culturing flask and waiting for detachment. 1 mL of cells was added to 10 mm×35 mm dishes with an additional 1 mL of medium. Cell concentration was approximately 1.5×105 cells/mL. To determine transfection, green fluorescence protein plasmid (GFP plasmid-pDRIVE5-GFP) was added to the medium 15 minutes before sonication. Various concentrations of GFP were used: 2 μg/mL, 15 μg/mL, and 30 μg/mL. The ultrasound contrast agent Definity, purchased from Lantheus Medical, was used to promote cavitation. The UCA volume used was 140 μL.

USD was performed using the Excel UltraMax therapeutic ultrasound machine, probe radius 2.5 cm. The ultrasound probe was coupled to the bottom of the cell dish using ultrasound gel. Ultrasound was applied for 60 seconds, at a 1 MHz frequency with varying output intensity: 0.3 W/cm2, and 0.5 W/cm2. The duty cycle was tested at 100% or 20% with a fixed pulse-repetition frequency of 100 Hz. As controls, we sonicated blank samples with no UCAs or GFP, and samples with GFP but no UCAs. Additionally, we ran a positive control using PEI, a lipofection agent. Finally, we prepared a sample that was not stimulated by ultrasound, but contained both Definity and GFP.

Cell counting was conducted in a fluorescence-activated cell-sorting (FACS) machine. 24 hours after USD, cells were collected in FACS test tube with 0.25% trypsin and washed once with 1×PBS. After all above, cells were resuspended in 200 uL 1% paraformaldehyde and tested through flow cytometry.

Cell viability was assessed by a cell count using a hemacytometer. After collecting cells in the FACS test tube, transfer 20 μl of each sample into small centrifuge tubes and dilute with 0.4% trypan blue. Put 10 μl in the hemacytometer and count cell number. Finally calculate the cell concentration with the following formula: Cell number counted in all squares/total number of squares counted*dilution factor*1×104.

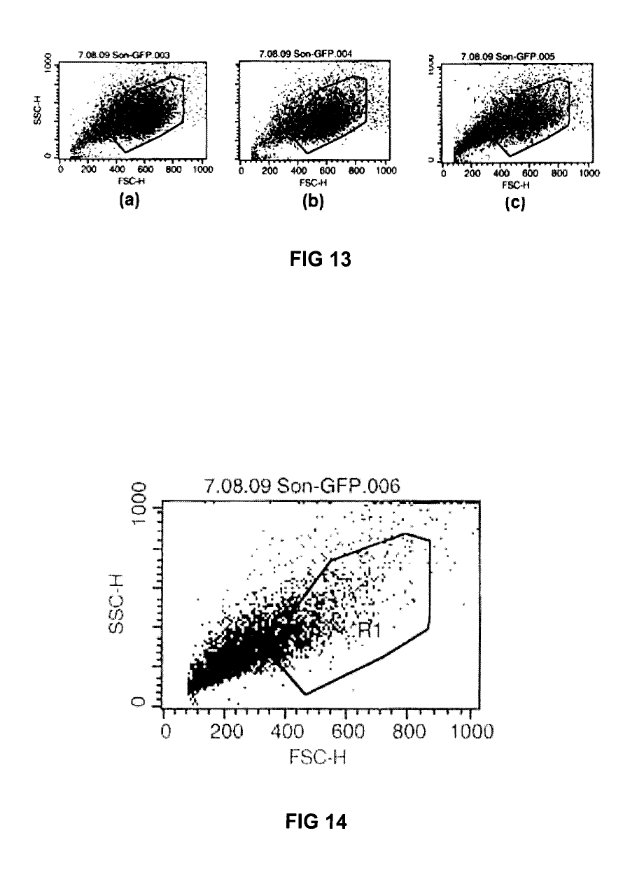

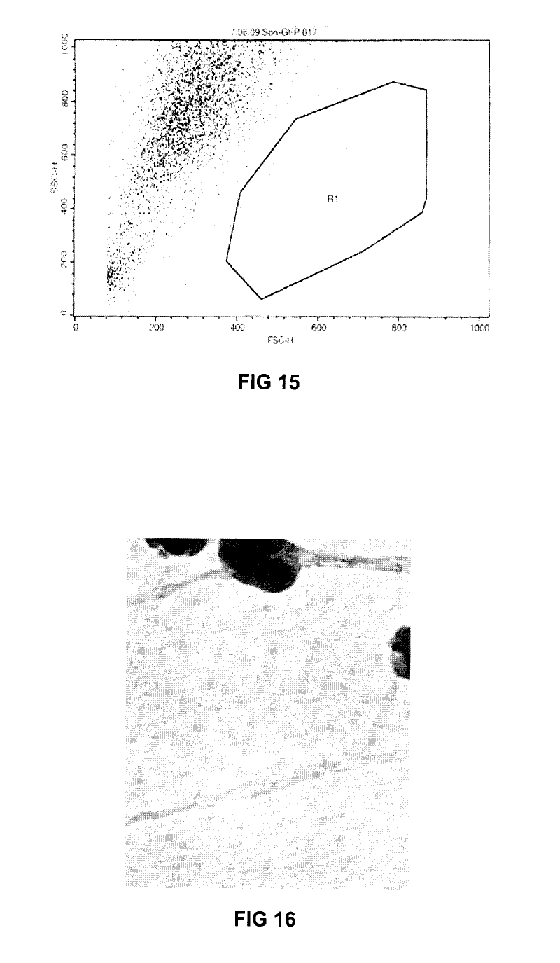

All the FACs test results are shown in FIGS. 12 to 15. Our negative control samples did not yield any transfection, but maintained excellent cell viability, as seen in the FACs result. The PEI lipofection positive control showed GFP expression, and extreme cell death.

TABLE 1
Transfection results of positive and negative control

| | GPF | Definity | | % |
|---|---|---|---|---|
| | [μg] | [μL] | W/cm²-DC-sec | Transfection |
| 1. | ◯ | 0 | 0-0-0 | 0.16% |
| 2. | 2 | 0 | 0-0-0 | 0.29% |
| 3. | 2 + PEI | 0 | 0-0-0 | 33.12% |

FACs analysis shows that as the exposure intensity increased the cell viability decreased. The maximum transfection was seen with an output intensity of 0.5 W/cm2 and a 20% duty cycle, at 32.51%. Cell viability is significantly lower at the output intensities above this level. This result suggests that the output energy achieved by a 0.5 W/cm2 and a 20% duty cycle, for 60 seconds is optimum for effective transfection.

The effect of DNA concentration on transfection efficiency was examined at every energy level. In every case, increasing the DNA concentration leads to an increase in transfection.

TABLE 2
Transfection results for varied ultrasound output
intensity, and GFP concentration.

| GFP | Definity | Output intensity, | Transfection |
|---|---|---|---|
| [μg] | [uL] | Duty cycle | % |
| 2 | 140 | 0.5 W/cm², 20% | 16.20% |
| 15 | 140 | 0.5 W/cm², 20% | 26.93% |
| 30 | 140 | 0.5 W/cm², 20% | 32.51% |
| 2 | 140 | 0.3 W/cm², 100% | 7.52% |
| 15 | 140 | 0.3 W/cm², 100% | 9.71% |
| 30 | 140 | 0.3 W/cm², 100% | 14.67% |
| 2 | 140 | 0.5 W/cm², 100% | 19.63% |
| 15 | 140 | 0.5 W/cm², 100% | 26.76% |
| 30 | 140 | 0.5 W/cm², 100% | 32.12% |

MCF-7 cells were used to evaluate the effects of ultrasound on gene delivery. We found that the efficiency of ultrasound mediated gene delivery, depended on plasmid concentration, while the viability of the cells was directly related to the ultrasound's output intensity. The latter could be due to the fact that the other physical effects of ultrasound, such as transient increase of local temperatures and pressure, are detrimental to cells, or that the pores the cavitation effect opened were unable to re-seal.

The results from the negative control samples show that the DNA plasmid GFP is unable to diffuse across the cell membrane on its own. The USD results show that the application of ultrasound with UCAs allow the DNA plasmid to transfect and be expressed by the cell. Furthermore, our results demonstrate that there is an optimum ultrasound exposure level for transfection and cell viability; the existence of optimum exposure parameters is consisted with other literary results. The FACs results exhibit that any output energy greater than 18000 mJ is detrimental to cell viability, where:

Energy(J)=Intensity*Duty Cycle*Time

Due to the nature of the FACs analysis, the transfection results obtained from the 0.5 W/cm2, 100% duty cycle sample may be skewed. Since a high percentage of cells in this sample were dead, transfection percentage we obtained is misrepresented and cannot be compared to our results obtained with higher cell viability.

Plasmid concentration was an important factor in determining transfection efficiency. In every case, transfection rate increased with DNA concentration. This result leads us to consider the importance of DNA proximity to the cells during USD. However, it is expected that the effect of increasing plasmid concentration to increase transfection efficiency will eventually plateau.

The findings from the lipofection agent, PEI, revealed two results. First, it confirms that the plasmid GFP can be expressed by the MCF-7 cells, but more importantly it highlights the importance of USD. The FACs results show an extremely high amount of cell death due to PEI. In contrast, USD was able to obtain similar transfection efficiency while maintaining a much lower cell death rate.

### Example 7

### Formation of Silica nanotubes

An amount of magnetic single-walled carbon nanotube powder was mixed with ground Na₂SiO₃.9H₂O (Na₂SiO₃.9H₂O/carbon nanotube ratio was 0.2 by volume). The mixture was ground carefully for 10 min to mix the reactants uniformly. Excessively ground NH₄Cl (NH₄Cl/Na₂SiO₃.9H₂O=3 by volume) was then added to the mixture. After being ground carefully for 50 min, the product was aged for 5 h and then washed three times with distilled water. Silicon dioxide coated nanotubes (Si-NT) were obtained after being dried at 60° C. for 5 h.

Particles core level spectra were measured using X-ray photoelectron spectrometer (VG ESCALAB MK II). The excitation source was a Mg X-ray anode and HV equalled to 20 eV.

To determine crystallite sizes and phase purity of the powders, the X-ray diffraction spectrum was obtained with a Rigaku D/max-rA X-ray diffractometer using Cu Kα (λ=1.54056 Å) radiation.

Si-NT’ morphology was observed with JEOL JEM 2010 transmission electron microscope (TEM) operating at 200 kV, as shown in FIG. 16. TEM samples were prepared by dispersing a small amount of powder in ethanol. A drop of the dispersion was then transferred onto coated grid and died for observation.

### Example 8

### Si-NT Functionalization

Oxidation of the Si-NTs: The Si-NTs (200 mg) were refluxed to introduce carboxylic groups. After refluxing, the solution was diluted with deionized water, filtered over a 0.2 μm polycarbonate filter (Millipore) and washed several times with deionized water. The sample was collected and dried overnight in a vacuum oven at 80° C. to give Si-NT-2 (170 mg).

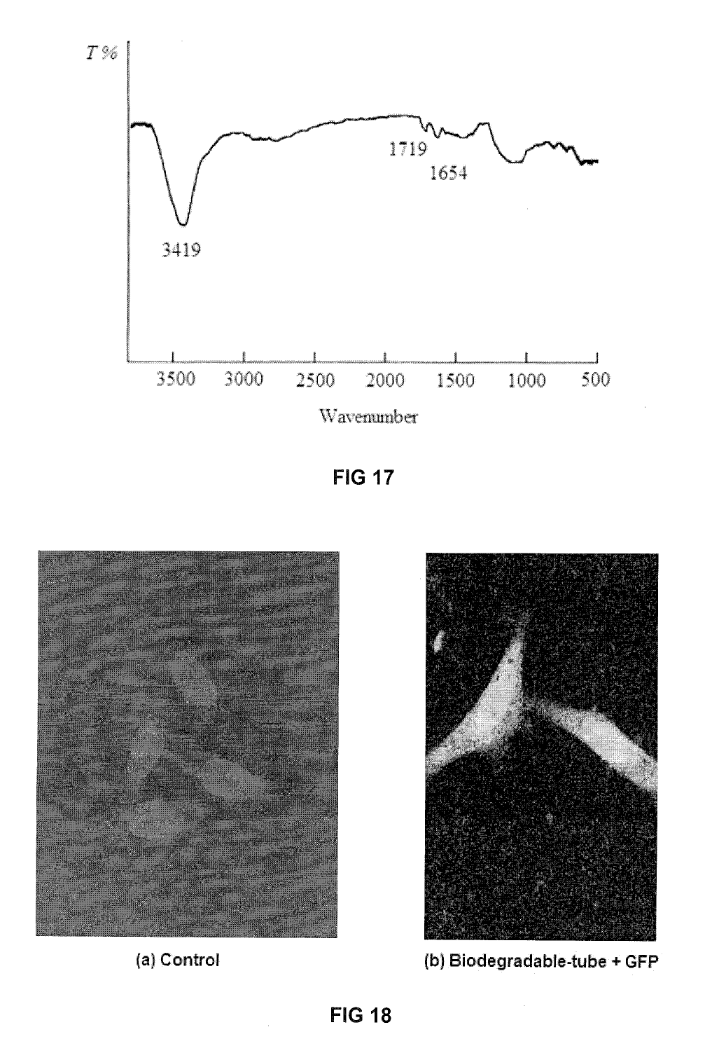

The carboxylated Si-NT underwent IR spectrum analysis, with the results shown in FIG. 17.

Reaction with thionyl chloride to give Si-NT-COCl: A suspension of Si-NT-2 (100 mg) in 20 mL of SOCl₂ together with 5 drops of dimethylformamide (DMF), was stirred at 70° C. for 24 h. The mixture was cooled and centrifuged at 2000 rpm for 30 min. The excess SOCl₂ was decanted and the resulting black solid was washed with anhydrous THF (3×20 mL), dried overnight in a vacuum oven at 80° C. to give Si-NT-3 (78 mg).

Coupling with ethylenediamine: The mixture of Si-NT-3 (50 mg) and anhydrous ethylenediamine (120 mL) was heated at 100° C. for 100 h. During this time, the liquid phase became dark. After cooling, the mixture was poured into methanol (100 mL), centrifuged to give a black solid, which was washed several times with methanol. The resulting solid was dried overnight in a vacuum oven at 80° C. to give Si-NT-4 (42 mg).

Functionalization with GFP plasmid: A suspension of the Si-NT-4 (25 mg) and GFP plasmid (5 mg) in anhydrous DMF (10 mL) was stirred in dark for 5 h, then the reaction mixture was poured into anhydrous ethyl ether (40 mL), centrifuged to give a black solid, which was washed with methanol until TLC (10% MeOH in dichloromethane) showed no free GFP left. The product was dried overnight in a vacuum oven at 80 0 C to get the final product (23 mg), Si-NT-GFP.

### Example 9

### Transfection of HeLa

HeLa cells were grown in RPMI 1640 supplemented with 10% FB in 35 mm Petri dish with a cover slip.

Si-NT-GFP solution was prepared by weighing 3 mg Si-NT-GFP powder into 50 ml centrifuge tube. 3 ml of sterilized DI water was added and sonicated until the silica tube powder dissolve and incubated for 1 hr at room temperature. The final volume was brought to 50 ml using RPMI 1640 medium w/o FBS. A similar solution with Si-NT was prepared as a control. The test and control silica tube solutions were added to 100 ml beakers.

200,000 cells were seeded per dish and cultured overnight allowing cells to attach. A volume of test or controls solutions were added to the dishes and the cells were then magnetically treated for 3 min vertically by putting dishes on top of magnetic stir hot plate and followed by 7 mins with Petri dishes on top of a stirring magnet.

The cells were washed twice with PBS, and replaced with 2 ml of culture medium. The dishes were returned to incubator and incubated for 24 hr and 48 hr, respectively.

Each of the samples were prepared for and viewed with confocal microscope observation of the GFP signal. The results are shown in FIG. 18.

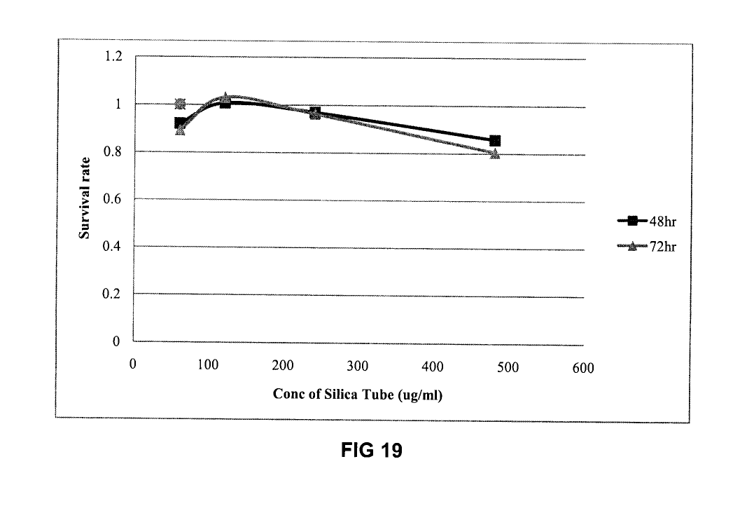

Toxicity studies showed that increasing concentrations of Si-NT had little effect on cell survival rate, as shown in FIG. 19.

### REFERENCES

The following references are representative of the level of skill in the art and are incorporated herein as if reproduced in their entirety (where permitted).
R. King, “Gene Delivery to Mammalian Cells by Microinjection”, from book “Gene Delivery to Mammalian Cells: Volume 1: Nonviral Gene Transfer Techniques”, ISBN: 978-1-58829-086-1
E. Heleniusi, M. Boije, V. Niklander-Teeri, E. Tapio Palva and T. H. Teeri, “Gene Delivery into Intact Plants Using the Helios™ Gene Gun”, Plant Molecular Biology Reporter, 18: 287a-2871, 2000
V F Tendeloo, P Ponsaerts, F Lardon, G Nijs, M Lenjou, C Broeckhoven, D R Bockstaele, Z N Berneman, “Highly efficient gene delivery by mRNA electroporation in human hematopoietic cells: superiority to lipofection and passive pulsing of mRNA and to electroporation of plasmid cDNA for tumor antigen loading of dendritic cells,” Blood. 2001 July 1; 98(1):49-56
H Pan, Y Zhou, F Sieling, J Shi, J Cui, C Deng, “Sonoporation of Cells for Drug and Gene delivery”, Engineering in Medicine and Biology Society, 2004. IEEE Conference on EMBS, Vol 2, September 2004 Page(s): 3531-3534
F Scherer1, M Anton, U Schillinger, J Henke, C Bergemann, A Kruger, B Gänsbacher and C Plank, “Magnetofection: enhancing and targeting gene delivery by magnetic force in vitro and in vivo”, Gene Therapy, January 2002, Volume 9, Number 2, Pages 102-109
A Watson and D Latchman, “Gene Delivery into Neuronal Cells by Calcium Phosphate-Mediated Transfection”, Methods, Volume 10, Issue 3, December 1996, Pages 289-291
G Beattie, E Goetzman, Q Tang, T Conlon, M Campbell-Thompson, D Matern, J Vockley, T R Flotte, “Recombinant adeno-associated virus-mediated gene delivery of long chain acyl coenzyme A dehydrogenase (LCAD) into LCAD-deficient mice”, The Journal of Gene Medicine, Volume 10 Issue 10, Pages 1113-1123, August 2008
T Bettinger, R Carlisle, M Read, M Ogris, and L Seymour, “Peptide-mediated RNA delivery: a novel approach for enhanced transfection of primary and post-mitotic cells”, Nucleic Acids Res. 2001 September 15; 29(18): 3882-3891.
Z Liu, A Fan, K Rakhra, S Sherlock, A Goodwin, X Chen, Q Yang, D, Felsher, H Dai, “Supramolecular Stacking of Doxorubicin on Carbon Nanotubes for in vivo cancer therapy”, Angew. Chem. Int. Ed., Volume 48, Issue 41, Pages:7668-7672, Sep. 28, 2009.
M Prato; K Kostarelos; A Bianco; C D. Partidos, “Biomedical applications of functionalized carbon nanotubes”, Chemical Communications, Volume 5, Pages 571-577, 2005.
Y. Sakakima, S. Hayashi, Y. Yagi, A. Hayakawa, K. Tachibana, and A. Nakao, “Gene therapy for hepatocellular carcinoma using sonoporation enhanced by contrast agents”, Cancer Gene Therapy (2005), 884-889
B. Patrick, P. C. Valerie, G. Adolfo, et al. “Naked DNA Injection for liver metastases treatment in rats”. Hepatology. 2002; 35:1144-1152.
Y. Yamashita, M. Shimada, K. Tachibana, et al. “In vivo gene transfer into muscle via electro-sonoporation”, Hum Gene Ther. 2002;13:2079-2084.
M. W. Miller, D. L. Miller, and A. A. Brayman. “A review of in vitro bioeffects of inertial ultrasonic from a mechanistic perspective”. Ultrasound Med. Biol. 22:1131-1154, 1996
T. Leighton, “The Acoustic Bubble”. Academic Press, San Diego, 1997 “Gene therapy progress and prospects: Ultrasound for gene transfer Revised and Expanded”, Marcel Dekker, Inc., pp 4
D. Dalecki, S. Z. Child, C. H. Raeman, C. Cox, E. L. Carstensen, “Ultrasonically induced lung hemorrhage in young swine”, Ultrasound Med Biol 1997a;23:777-781.
P. E. Huber, P. Pfisterer, “In vitro and in vivo transfection of plasmid DNA in the Dunning prostate tumor R3327-AT1 is enhanced by focused ultrasound”. Gene Ther 2000;7:1516-1525.
H. D. Liang, Q. L. Lu, S. A. Xue, and M. Halliwell “Optimization of Ultrasound-mediated Gene Transfer (Sonoporation) in Skeletal Muscle Cells”,
T. Kodama, D. O. Cosgrove, H. J. Stauss, T. A. Partridge and M. J. K. Blomley, Ultrasound in Med. & Biol., Vol. 30, No. 11, pp. 1523-1529, 2004

1. A method of delivering a molecule across a cell membrane using a delivery vehicle comprising a molecule fixed on a magnetic nanoparticle, the method comprising the steps of:
(a) positioning the nanoparticle in the immediate vicinity of the cell membrane; and
(b) simultaneously subjecting the nanoparticle and cell membrane to magnetic field and ultrasound.

2. The method of claim 1 wherein the ultrasound comprises low-intensity pulsed ultrasound.

3. The method of claim 1 wherein the nanoparticle comprises a single-walled carbon nanotube.

4. The method of claim 1 wherein the nanoparticle comprises a biodegradable or biocompatible material.

5. The method of claim 4 wherein the nanoparticle comprises silica.

6. The method of claim 1 wherein the molecule comprises a DNA or a RNA molecule.

7. The method of claim 1 which is practiced in vivo, and wherein the delivery vehicle are concentrated in a specific region by a magnetic force placed adjacent the specific region, and forced across a cell membrane by a magnetic field.

8. The method of claim 7 wherein the magnetic field alternates direction across the specific region after concentration of the delivery vehicle.

9. The method of claim 7, wherein the specific region is behind or associated with the blood-brain barrier.
